(19) United States
(12) Patent Application Publication
(10) Pub. No.: US 2022/0183878 A1
(43) Pub. Date: Jun. 16, 2022

(54) URINE DRAINAGE APPARATUS FOR WOMEN TO USE WHILE LYING DOWN OR SITTING

(71) Applicant: MEDHlBlT MEDlKAL SAGLIK BlLlSlM LlMlTED SlRKETl, Kocaeli (TR)

(72) Inventors: Ebru Güler, Karslyaka/lzmir (TR); Sermet Haldun Güler, Karslyaka/lzmir (TR)

(73) Assignee: MEDHlBlT MEDlKAL SAGLIK BlLlSlM LlMlTED SlRKETl, Kocaeli (TR)

(21) Appl. No.: 17/437,967

(22) PCT Filed: Apr. 16, 2019
(86) PCT No.: PCT/TR2019/050246

§ 371 Date: Sep. 10, 2021

(30) Foreign Application Priority Data
Mar. 19, 2019 (TR) 2019/04100

(51) Int. Cl. A61F 5/455; A61F 5/44

(52) U.S. Cl. CPC A61F 5/4556; A61F 5/4404

(57) ABSTRACT

This invention relates to a urine drainage apparatus equipped with a mouth portion ensuring the apparatus to fit on the area of use, ensuring sealing with its inner and outer angles and ensuring urine drainage in the lying down or sitting position with help or alone.

### TECHNICAL FIELD

This invention relates to a urine drainage apparatus used for the drainage of the urine of women and girls who are bedridden or do not have access to the toilet.

### STATE OF ART

The flow direction of the women's urine requires the women to sit down while urinating. This creates a problem for women when they are faced with a situation where they cannot sit down. For example,
When there is no toilet seat cover or when sitting on the toilet seat is not desired,
When they have to drain the urine with the help of a caregiver while lying down in the hospital or at home,
When there is no caregiver and the bedridden patient has to urinate alone
Women and girls who are bound to a wheelchair and have difficulty going to the toilet,
Women and girls who are stranded in traffic and have no chance of getting out of the car,
Women and girls who cannot use the toilet due to hygienic reasons,
Women (brides) who need to go to the toilet but cannot go to the toilet because of their clothes,
Women who often wake up to use the toilet at night but at risk to go to the toilet alone in the night (due to falls, dizziness, etc.),
Women with a problem in their feet who need to go to the toilet but will not be able to reach in time,
Women hiking in nature, camping have difficulty in these situations,
For women who are bedridden for certain reasons, known methods do not provide a solution for urine drainage.

No special product has been developed for the urine drainage of bedridden patients and there are products such as ducks and sliders in the market. The duck is generally produced for men and the version allegedly produced for women is the same. This product is uncomfortable to use, lacks functions, and requires the change of bed sheets due to leaks. The slider is actually made for feces and when used for urine drainage, it causes leakage due to its use and requires the change of bed sheets, and also causes pain during placement for the bedridden patient. Considering that the bedridden patient usually consumes fluids, changing bed sheets or bed protectors more than 4-5 times a day is both very painful and very inconvenient for the caregiver. Our product provides ease of use for both the patient and the caregiver and also solves the leakage problem.

Although patient diapers or underpants seem to be more hygienic and comfortable than sliders and ducks, they need to be changed frequently to prevent diaper rash and infections, they are not cost-effective solutions since they are disposable. Since patient diapers or underpants come into contact with the skin, they cause diaper rash and infection and even open wounds depending on the contact time. Our product prevents such problems because it is used and removed.

In some cases, catheters are placed for urinary drainage to the women unable to move (in cases such as a hip fracture) and this causes unnecessary infection risk and discomfort for the patient.

The patent document with application Ser. No. 12/495,399 discloses a urine drainage apparatus. The present invention includes a tube with packaging for an extra labial fluid drainage apparatus, a cylindrical wall, a closed end, and an open end. The cylindrical wall and the closed end define a cavity which may include the discharge apparatus. Cylindrical wall is flexible and has an inner diameter. A cap is adapted to close and open the tube at the open end, respectively. The cap has an end plate with a circular flange extending from it. The annular flange is rigid and has an outer diameter. The annular flange can be placed in the cavity in the open end of the tube and inside the inner diameter of the cylindrical wall.

The outer diameter of the flange is larger than the inner diameter of the cylindrical wall, and the outer diameter and the inner diameter can be changed into a press fit embodiment when the cap is connected to the pipe. The angled structure of the apparatus according to the invention is used for urine drainage while standing up and does not offer a solution for urine drainage in bed and does not have a suitable mouth structure for this.

The patent document with application Ser. No. 15/15,582 is related to a female urinal apparatus. The disposable female urinal comprises a funnel-shaped body made of a molded fiber material. The body comprises a relatively wide receiving opening at one end which narrows along the length of the body, narrowing one end to an outlet opening forming a mouth. The receiving mouth is surrounded by a sealing edge having a shape configured to be pressed against the user's crotch region. The rim includes a curved rotating edge such that the exposed end surface of the molded fiber body is directed away from the front end of the device facing the user during use and such that it will not come into contact with the user when the edge is pressed against the device. The invention is also a suitable apparatus for urine drainage while standing, and all of its features will make it easier to drain urine while standing, and in case it is used while lying down, it will cause leakage due to its structure.

### DESCRIPTION OF THE INVENTION

The present invention completely overcomes the aforementioned drawbacks with its mouth portion that allows the apparatus to fit on the area of use and the inner and outer angles of the rest of the apparatus.

The invention is a drainage apparatus which can be used repeatedly and includes a funnel-like body made of molded thermoplastic materials.

The portion fitting on the body is wide, the outlet portion is narrower so that it can be inserted into the apparatus suitable for collecting the urine drainage.

The mouth fitting on the body is surrounded by a sealing edge configured with a suitable material to press onto the user's crotch area.

The invention consisting of thermoplastic material is manufactured at once in one piece, but with two separate materials using plastic injection method. The portion that is in contact with the body and called lip is made of TPE and the other parts of the apparatus are made of Polyethylene material. Both materials are manufactured from one mold at once, fused together, ensuring integrity.

The urine output section of the product has a structure which can connect with the catheter bag, which is a standard material used all over the world.

The catheter tube is inserted so that it enters the outlet, which has an internal diameter of 10 mm. In the absence of a catheter, rubber tubes with a diameter between 12 and 20 mm can be easily mounted on the outlet. The length of the rubber tube completely depends on the wishes of the user. Additionally, the end can enter any plastic bottle or bucket-like product and urine can be collected there.

### DRAWINGS

The invention will now be described with reference to the accompanying drawings, whereby the features of the invention will become more apparent and appreciated, but this is not intended to limit the invention to these particular embodiments. On the contrary, it is intended to cover all alternatives, modifications, and equivalents which may be included in the scope of the invention as defined by the appended claims. It should be understood that the details shown are provided solely for the purpose of illustrating the preferred embodiments of the present invention and providing the most useful and easily understandable definition of both the methods and the rules and conceptual features of the invention. In these drawings;

FIG. 1 External curve of the urine drainage apparatus

FIG. 2 Internal curve of the urine drainage apparatus

### DESCRIPTION OF THE REFERENCES

1 Peak

2 Outer curve starting point

3 Outer curve center

4 Closing curve start

5 Closing curve end

6 Back curve start

7 Back curve center

8 Back curve

9 Back curve end

10 End point

11 Lip end point

12 Inner curve start

13 Rear section curve

14 Rear section curve end

15 Inner curve center

16 Inner curve end

17 Lip front end

### DESCRIPTION OF THE DETAILS IN THE DRAWINGS

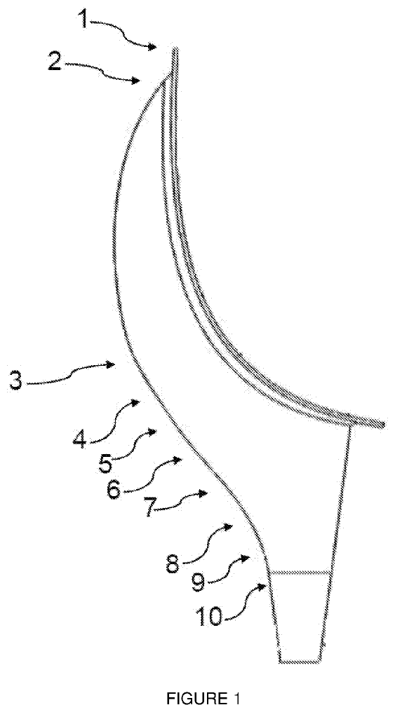

FIG. 1 shows the outer curve structure of the urine drainage apparatus. The apparatus has a shape similar to an “S” with Radius in the most appropriate angles to provide the urine drainage in both the lying position and the sitting position.

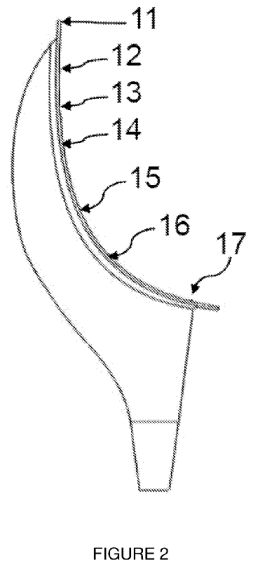

FIG. 2 shows the inner curve structure of the urine drainage apparatus. Since this section is in contact with the body, the sections in contact with the body form a lip structure. This lip structure progresses downwards with special Radius angles to ensure sealing. Thus, urine output is provided without leakage and does not cause any irritation in the body and also allows the urine output in the lying position.

### DETAILED DESCRIPTION OF THE INVENTION

The outer curve begins with the peak (1). The peak (1) is the start of the portion which is in contact with the rear of the area used during the use of the urine drainage apparatus. This portion includes the outer curve starting point (2) and the outer curve center (3) sections. As this is the reverse side of the flow direction of the urine, it has Radius openings which prevent the urine from running to this area and overflowing or leaking from this area. The curved structure between the outer curve peak (1) and the outer curve center (3) has angles that allow the urine to be pushed forward, not backward, and to adjust the shape of the apparatus in lying position so as not to obstruct the drainage process and also to collect the urine flowing backward and push it forward without causing leakage.

The outer curve peak (1) angles are in R3-R4 range. The subsequent outer curve start point 2 has angles between R63 and R65 and these angles continue increasing up to the outer curve center (3). The outer curve center (3) angles are in R137-R139 range. The section between the outer curve start point (2) and the outer curve center (3) is optimally increased from R64 to R138.2.

The outer curve structure proceeds with an angle form directed inward from the closing curve start (4) to the closing curve end (5), and thanks to this form, it allows the urine to accumulate some in such way that it does not cause leakage. The closing curve start 4 has an angle between R58 and R59 and the optimum angle is R58.2. The closing curve starts (4) portion continues up to the closing curve end (5). The angle value decreasing up to this point decreases and the angle value at the end of the closing curve end (5) is between R54.5 and R56. The optimum value of the angle value at the closing curve end (5) is defined by the angle R55.5.

In the continuation of the closing curve end (5) point, the angles outer portion of the apparatus is determined to direct the urine output and not to prevent the urine output in the lying position and to prevent any obstructions due to crushing. The back curve start (6), therefore, has an inward direction and has angle values between R94-R94.5 and the optimum angle is R94.2. In its continuation, the angle value is increased up to R371.7, which is the optimum value, at the back curve center (7). This increase in this value facilitates the urine output. It has an angle between R370 and R372. In the continuation of the back curve center (7), the angle value decreases to R202.1 and it increases to R663.7 at the back curve (8) point. Then, the angle value decreases to R81.6 angle value at the back curve end (9). At the end point (10), the angle value decreases from the curve end 9 down to R43.6.

The inner part of the urine drainage apparatus in contact with the body has a lip structure its curved structure. This curved structure has angles to provide a sealed contact with the body. The lip end point (11) starts with an angle of R544.4 and the angle value continues to decrease with the curved structure of this part of the body. At the inner curve start (12), this angle decreases to R303.1 and then decreases to R199.5 at the rear section curve (13). The rear section curve end (14) continuing with the rear section curve decreases to an angle value of R131.4. Continuing to the inner curve center (15), the angle value decreases to R83.7 at the inner curve center (15). At the inner curve end (16), the angle value decreases to R58.6. At the lip front end (17), the angle value increases to R116.3 to prevent leakage.

Additionally, at the urine output, the catheter tube is inserted so that it enters the outlet, which has an internal diameter of 10 mm. In the absence of a catheter, rubber tubes with a diameter between 12 and 20 mm can be easily mounted on the outlet. The length of the rubber tube completely depends on the wishes of the user. Additionally, the end can enter any plastic bottle or bucket-like product and urine can be collected there.

What is claimed is:

1. A urine drainage apparatus for women allowing during drainage in both sitting and lying down positions; characterized in that; ensuring the urine flow and accumulation and preventing any leakages in sitting or lying down positions, the outer portion has the following characteristics:
the outer curve peak (1) angles are in R3-R4 range and the optimum angle is R3.5,
the outer curve start point (2) has angles between R63 and R65 and these angles continue increasing up to the outer curve center (3),
the outer curve center (3) angles are between R137 and R139, and the optimum angle is R138.2,
it proceeds with an angle form directed inward from the closing curve start (4) to the closing curve end (5), and the closing curve start (4) has angle values between R58 and R59, and the optimum value is R58.2, and this angle value decreases towards the closing curve end (5) and the angle value at the closing curve end (5) is between R54.5 and R56, and the optimum value is R55.5,
the back curve start (6) has an inward direction and has angle values between R94-R94.5 and the optimum angle value is R94.2,
in its continuation, the angle value increases up to a value between R370 and R372 at the back curve center (7),
in the continuation of the back curve center (7), the angle value decreases to R202.1 and it increases to R663.7 at the back curve (8) point,
then, the angle value decreases to R81.6 angle value at the back curve end (9), and
at the end point (10), the angle value decreases from the curve end (9) down to R43.6.

2. A urine drainage apparatus for women allowing during drainage in both sitting and lying down positions; characterized in that; allowing the portion in contact with the body have a sealed contact with the body suitable for the anatomical structure and maintaining this sealing in the lying down position, the inner section is equipped as follows:
at the inner curve start (12), this angle decreases to R303.1 and then decreases to R199.5 at the rear section curve (13),
the rear section curve end (14) continuing with the rear section curve decreases to an angle value of R131.4,
continuing to the inner curve center (15), the angle value decreases to R83.7 at the inner curve center (15), and
at the inner curve end (16), the angle value decreases to R58.6.

3. The urine drainage apparatus of claim 2, characterized in that, to prevent leakage the end portions are equipped as follows:
the lip end point (11) starts with an angle of R544.4 and the angle value continues to decrease with the curved structure of this part of the body, and
at the lip front end (17), the angle value increases to R116.3 to prevent leakage.

4. The urine drainage apparatus of claim 1, comprising an external apparatus such as a catheter bag or a rubber tube mounted on the outlet of the drainage.

5. The urine drainage apparatus of claim 2, comprising an external apparatus such as a catheter bag or a rubber tube mounted on the outlet of the drainage.

6. The urine drainage apparatus of claim 3, comprising an external apparatus such as a catheter bag or a rubber tube mounted on the outlet of the drainage.
